(19) United States
(12) Patent Application Publication
(10) Pub. No.: US 2017/0080091 A1
(43) Pub. Date: Mar. 23, 2017

(54) GEL SHEET USING HYALURONIC ACID

(71) Applicant: BioPlus Co., Ltd., Seoul (KR)

(72) Inventor: Sung Young PARK, Daejeon (KR)

(21) Appl. No.: 15/272,438

(22) Filed: Sep. 22, 2016

(30) Foreign Application Priority Data
Sep. 22, 2015 (KR) 10-2015-0133753

(51) Int. Cl. A61K 47/36; A61K 47/12; A61K 9/06

(52) U.S. Cl. CPC A61K 47/36; A61K 9/06; A61K 47/12

(57) ABSTRACT

A gel sheet, prepared by immersing a primary hyaluronic acid gel sheet, which is prepared by drying a flat plate on which an aqueous solution obtained by dissolving an alkali salt of high-molecular-weight hyaluronic acid in an alkaline solution is applied in a vacuum oven, in a crude solution of a low-molecular-weight organic acid anhydride or an anhydride solution diluted with a unit organic acid forming the anhydride, and secondarily drying the immersed primary hyaluronic acid gel sheet in the vacuum oven.

### CROSS-REFERENCE TO RELATED APPLICATION

This application claims priority to and the benefit of Korean Patent Application No. 10-2015-0133753, filed on Sep. 22, 2015, the disclosure of which is incorporated herein by reference in its entirety.

### BACKGROUND

1. Field of the Invention

The present invention relates to a gel sheet with increased mechanical strength and biocompatibility and a method therefor, using hyaluronic acid.

2. Discussion of Related Art

Hyaluronic acid is a linear polymer including alternatively bonded β-N-acetyl-D-glucosamine and β-D-glucuronic acid, and is a polysaccharide commonly having a molecular weight in the range of 50,000 to 10,000,000 Da or more. Hyaluronic acid is a basic material of connective tissue in vivo, and mainly distributed in the skin of a mammal, synovial fluid, the vitreous humor, the umbilical cord, serum, the cock's comb, etc. and also known to be present in capsules of streptococci or the like. Examples of general methods for preparing hyaluronic acid include a method of extracting hyaluronic acid form the cock's comb, umbilical cord and the like, a method of culturing streptococci of Lancefield groups A and C, genetically recombined Bacillus subtilis or the like and then extracting hyaluronic acid therefrom for refinement, etc.

Natural hyaluronic acid is polydisperse in terms of molecular weight, and shows excellent biocompatibility when transplanted or injected into the body regardless of its origin due to not having species specificity and tissue or organ specificity, and thus is being used for ophthalmic injections.

However, since hyaluronic acid is easily decomposed by an enzyme called hyaluronidase that is present in vivo, the in vivo half life of hyaluronic acid is about 0.5 to 3 days and the retention time in the body is relatively short, and thus the direct use of a natural polymer in biomedical materials or the like has limitations. Consequently, in order to extend its use to biomedical materials, there has been an attempt to increase in vivo sustainability using a method of modifying hyaluronic acid by crosslinking hyaluronic acid or bonding a functional group using various types of chemical modifiers.

A representative one among them is a crosslinked hyaluronic acid gel with high swellability obtained using a bifunctional crosslinking agent such as divinyl sulfone, bis-epoxide or formaldehyde (U.S. Pat. No. 4,582,865, JP1994-037575 and JP3107488).

A method of chemical modification of hyaluronic acid using the solubility of tetrabutylammonium hyaluronate in an organic solvent such as dimethyl sulfoxide (DMSO) (JP-A-3-105003) has also been suggested. There has been proposed a method of forming ester linkages between the carboxyl groups and the hydroxyl groups in hyaluronic acid by treating tetrabutylammonium hyaluronate with triethylamine and 2-chloro-1-methylpyridinium iodide in DMSO (EP-A-0341745A1).

Further, as an approach to insolubilization of hyaluronic acid in water without using covalently binding chemicals, a method of preparing a hyaluronic acid-polymer complex by ionically bonding hyaluronic acid and a polymer having an amino or imino group via the carboxyl groups in hyaluronic acid and the amino or imino group in the polymer has been suggested (JP-A-6-73103).

In addition, a method using a carbodiimide or a succinimidyl active ester as a crosslinking agent has been proposed (WO94/2517, U.S. Pat. No. 4,970,298).

### SUMMARY OF THE INVENTION

It is an objective of the present invention to provide a gel sheet with increased mechanical strength and biocompatibility and a method therefor, using hyaluronic acid.

A gel sheet according to still another embodiment of the present invention may be prepared by immersing a primary hyaluronic acid gel sheet, which is prepared by drying a flat plate on which an aqueous solution obtained by dissolving an alkali salt of high-molecular-weight hyaluronic acid in an alkaline solution is applied in a vacuum oven, in a crude solution of a low-molecular-weight organic acid anhydride or an anhydride solution diluted with a unit organic acid forming the anhydride, and secondarily drying the immersed primary hyaluronic acid gel sheet in the vacuum oven.

The anhydride solution diluted with a unit organic acid forming an anhydride may have a concentration (v/v) in a range of 5 to 100 vol %.

The immersion step may be performed at 4 to 70° C. for 2 hours or more.

The anhydride may include acetic anhydride, propionic anhydride, butyric anhydride, caproic anhydride and isobutyric anhydride.

The vacuum oven may have a predetermined degree of vacuum and a predetermined temperature before the secondary drying.

The immersed primary hyaluronic acid gel sheet may be transferred into an incubator maintained at 40° C. before the secondary drying.

The predetermined degree of vacuum may be 100 Torr or less.

The temperature of the vacuum oven and a primary drying time may be gradually increased during the primary drying.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a flow chart illustrating a method for preparing a gel sheet using hyaluronic acid according to an embodiment of the present invention.

### DETAILED DESCRIPTION OF EXEMPLARY EMBODIMENTS

The particular structural or functional descriptions of embodiments according to the concepts of the present invention disclosed in the specification or the application are only intended for the purpose of describing embodiments according to the concepts of the present invention and the embodiments according to the concepts of the present invention may be practiced in various forms and should not be construed as being limited to those described in the specification or the application.

Since the present invention can be subjected to various modifications and have several embodiments, particular embodiments will be illustrated in the drawings and described in the detailed description in detail. However, it is not intended to limit the present invention to particular embodiments and it should be understood that the present invention covers all modifications, equivalents, and/or replacements that fall within the spirit and technical scope of the present invention.

Although the terms “first” and “second” may be used to describe various components, these components should not be limited by these terms. The terms may be used only for the purpose of distinguishing one component from another component, e.g., a first component may be named a second component without departing from the scope of right according to the concepts of the present invention and similarly, a second component may also be named a first component.

When any component is referred to as being ‘connected’ to another component, it should be understood that the former can be ‘directly connected’ to the latter, or there may be another component therebetween. On the contrary, when any component is referred to as being ‘directly connected’ to another component, it should be understood that there may be no other component therebetween. Other expressions describing the relationship between components, such as “between” and “directly between” or “adjacent to” and “adjacent directly to” should be also construed in the same way.

The terms used herein are only used to describe specific embodiments and not intended to limit the present invention. In the following embodiments, the terms in singular form may include the plural form unless otherwise specified. It should be understood that the terms “includes” or “has” indicate the presence of characteristics, numbers, steps, operations, components, parts or combinations thereof represented in the present disclosure but do not exclude the presence or addition of one or more other characteristics, numbers, steps, operations, components, parts or combinations thereof.

Unless otherwise defined herein, all terms used herein including technical or scientific terms have the same meanings as those generally understood by a person skilled in the art. Terms defined in dictionaries generally used should be construed to have meanings matching with contextual meanings in the related art and are not to be construed as having an ideal or excessively formal meaning unless otherwise defined herein.

Hereinafter, embodiments will be described in detail by explaining exemplary embodiments of embodiments with reference to the attached drawings. Like reference numerals in the drawings denote like elements.

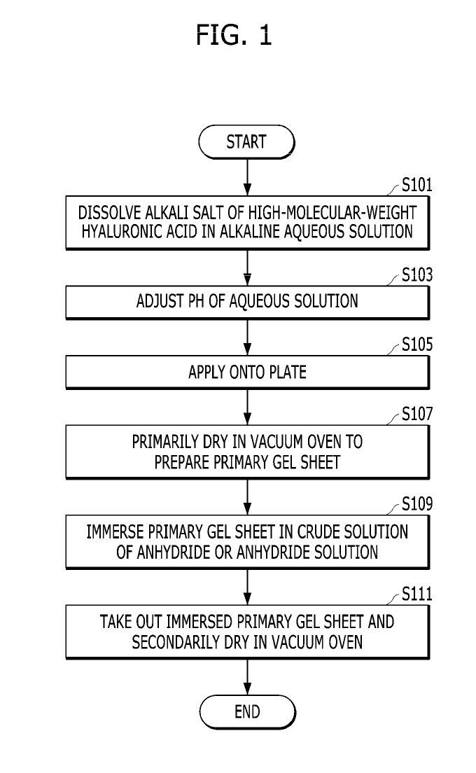

FIG. 1 is a flow chart illustrating a method for preparing a gel sheet using hyaluronic acid according to an embodiment of the present invention.

The objective of the present invention is to prepare a gel sheet using hyaluronic acid which can maintain its shape for a long time, exhibit improved mechanical strength, and have a low biodegradation rate due to not being dissolved in an aqueous solution with a pH from 5.5 to 8.5 that is a common in vivo condition, a physiological saline solution or a buffer solution, and high biocompatibility. The subject of this preparation method may be a preparation device.

The gel sheet using hyaluronic acid is prepared according to the following steps.

Alkali salts of high-molecular-weight hyaluronic acid having a molecular weight in a range of 50,000 to 4,000,000 Da are dissolved in an alkaline solution with a pH from 9.0 to 14.0 to prepare an aqueous solution having a concentration (w/v) in a range of 1 to 10 wt % (S101). The pH of this aqueous solution is adjusted to 5.5 to 8.5 (S103), and the aqueous solution is thinly applied onto a plate such as a glass plate or a plastic plate (S105).

Examples of the alkali salts of hyaluronic acid include sodium salts or calcium salts of hyaluronic acid, and water and hyaluronic acid have to form a hydrogen bond such that these alkali salts of hyaluronic acid are dissolved in water. Accordingly, when the pH of water is adjusted to an alkaline state, sodium or potassium forming salts by binding to hyaluronic acid is easily disassociated such that the formation of the hydrogen bond of water and hyaluronic acid can occur well, thereby obtaining the effect of accelerating the dissolution rate of hyaluronic acid.

It is preferable to increase the alkalinity of water in proportion to the concentration of hyaluronic acid to be dissolved. However, it is preferable to adjust the concentration of hyaluronic acid to pH 9.0 to 12.0 because an excessively high pH may result in hydrolysis of hyaluronic acid. Sodium hydroxide or potassium hydroxide may be used to adjust the alkalinity of water (alkaline solution).

When the hyaluronic acid is fully dissolved, the pH of the hyaluronic acid aqueous solution may be adjusted to 5.5 to 8.5, and more suitably to 7.4 using a dilute hydrochloric acid solution. When the pH of the hyaluronic acid aqueous solution is not adjusted to be nearly neutral, hyaluronic acid is likely to be decomposed since hydroxide ions in the hyaluronic acid aqueous solution are concentrated to drastically increase the pH when moisture is evaporated by subsequent drying in a vacuum oven. Consequently, it is preferable to adjust the pH of the hyaluronic acid aqueous solution to be nearly neutral before drying in a vacuum oven.

The pH-adjusted hyaluronic acid solution is applied onto a glass plate or a plastic plate with a smooth surface. Here, the higher the concentration of the hyaluronic acid solution is, the thicker the thickness of the applied solution is.

Thereafter, the hyaluronic acid solution thinly applied on the plate is dried in a vacuum oven at a predetermined temperature to prepare hyaluronic acid gel sheet (S107). The drying time is 12 hours or more, and may be adjusted to be shorter as the predetermined temperature is higher.

The thickness of the prepared hyaluronic acid gel sheet may vary with the concentration of the hyaluronic acid solution. When the concentration of the hyaluronic acid solution is high, a harder and denser gel sheet may be prepared.

When the predetermined temperature in a vacuum oven is set high, a small amount of dry skin is formed during drying of a gel sheet, a drying speed is high and mechanical strength is relatively low. On the other hand, when the predetermined temperature in a vacuum oven is set low, a large amount of dry skin is formed during drying of a gel sheet, a drying speed is low and mechanical strength is relatively high.

After drying is complete, the dried hyaluronic acid gel sheet is immersed in a crude solution of a low-molecular-weight organic acid anhydride or a diluted solution diluted with a unit organic acid forming the anhydride to have a concentration (v/v) from 5 to 100 vol % (S109), and maintained at 4 to 70° C. for 2 to 120 hours.

An anhydride denotes an anhydride of a low-molecular-weight organic acid, and the low-molecular-weight organic acid is preferably widely present in a biological system. The type of the anhydride is not limited, but examples thereof may include acetic anhydride, propionic anhydride, butyric anhydride, caproic anhydride and isobutyric anhydride.

This anhydride may increase the strength of the hyaluronic acid gel sheet by promoting a condensation reaction. An anhydride may adjust its concentration (vol %) by mixing with a unit organic acid forming the anhydride. For example, acetic anhydride may be diluted with acetic acid, propionic anhydride may be diluted with propionic acid, butyric anhydride may be diluted with butyric acid, and an anhydride is preferably diluted to have a concentration from 5 to 100 vol %.

The hyaluronic acid gel sheet is taken out of the crude solution of a low-molecular-weight organic acid anhydride or the diluted solution diluted with a unit organic acid forming an anhydride, and is then introduced into a vacuum oven maintained at the predetermined degree of vacuum and the predetermined temperature (S111). Through this step, residual anhydrides are eliminated to prepare a nearly-white transparent hyaluronic acid gel sheet with enhanced mechanical strength.

The predetermined degree of vacuum of the vacuum oven has to be 100 Torr or less on the basis of an absolute pressure for completely removing the residual low-molecular-weight organic acid and anhydride thereof from the hyaluronic acid gel sheet. The predetermined temperature in the vacuum oven may range from 60 to 180° C. The hyaluronic acid gel sheet taken out of the crude solution or diluted solution of an anhydride may be dried in the vacuum oven for 2 hours or more, and preferably for 12 hours or more.

When the temperature in the vacuum oven is drastically increased in the state where the degree of vacuum in the vacuum oven is insufficient, the hyaluronic acid gel sheet may be degraded, and thus the temperature in the vacuum oven is increased after the degree of vacuum is increased at room temperature to reach 100 Torr or less which is the predetermined degree of vacuum. When the temperature in the vacuum oven becomes high, the energy necessary for a condensation reaction can be easily provided, resulting in the effect of increasing the mechanical strength of the hyaluronic acid gel sheet.

Hereinafter, the present invention will be described in further detail with reference to examples and comparative examples. However, the following examples and comparative examples are for illustrative purposes only and not intended to limit the scope of the present invention.

### Examples 1 to 4

0.24 g of sodium hyaluronate having a molecular weight of approximately 1,300,000 Da converted into the intrinsic viscosity of 1.8 m³/kg was dissolved in 20 ml of a sodium hydroxide solution with a concentration of 0.05 mol/l at room temperature to prepare an aqueous solution having a concentration of 1.2 wt %.

The pH of the hyaluronic acid aqueous solution was adjusted to pH 7.4 using a dilute hydrochloric acid solution. 5 ml of each of the pH-adjusted hyaluronic acid aqueous solution was applied onto four Petri dishes with a diameter of 60 mm such that the aqueous solution had a thickness of about 2 mm. These Petri dishes were all transferred to a vacuum oven and the pressure of the vacuum oven was maintained at 100 Torr or less by reducing the pressure.

The temperature of the vacuum oven was set to 60° C. and maintained for 120 minutes. The temperature of the vacuum oven was raised to 80° C. and maintained for 240 minutes. The temperature of the vacuum oven was raised to 120° C. and maintained for 18 hours, and thereby milky-white gel sheets were primarily prepared after drying.

The primarily prepared hyaluronic acid gel sheets were introduced into 15 ml of each of an acetic acid anhydride solution having a concentration of 5, 20, 50 and 100 vol % as presented in the following Table 1. More precisely, the anhydride solutions were introduced into the Petri dishes containing the primarily prepared hyaluronic acid gel sheets and retained in an incubator maintained at 40° C. for 24 hours. Thereafter, the Petri dishes were transferred to a vacuum oven, and heating of the vacuum oven was started when the degree of vacuum reached 100 Torr or less by reducing the pressure of the vacuum oven.

The temperature of the vacuum oven was set to 100° C. and maintained, and nearly-white transparent secondary hyaluronic acid gel sheets were prepared after 24 hours. The secondary hyaluronic acid gel sheets thus prepared were all cut in half and each was dipped in buffer solutions with a pH of 5.5 and 7.4. Then, the solutions were carried into an incubator maintained at 40° C. and observed at 24-hour intervals for 168 hours to determine whether or not the secondary hyaluronic acid gel sheet was maintained and the degree of degradation.

The four examples all showed that the shape of the gel sheet was maintained well and no sheet was degraded and disintegrated. The results thus determined are presented in the following Table 1.

Furthermore, the cytotoxicity was confirmed as follows. 50 mg of each of the secondary hyaluronic acid gel sheets was mechanically pulverized using a homogenizer. Each of them was mixed with 2 ml of a cell culture medium (DMEM including 10 vol % of fetal bovine serum) and maintained in a cold room (4° C.) for 7 days, such that components of the hyaluronic acid gel sheets were sufficiently extracted.

After 7 days, the gel sheets were eliminated by centrifugation and supernatants were only obtained, and each of the supernatants was introduced into a 6-well dish.

Each well was inoculated with 1×10⁴ cells to start the cultivation, and a cell density was observed after 2, 4, 6 and 8 days from starting of the cultivation to confirm the cytotoxicity.

A cell population proliferating in DMEM including 10 vol % of fetal bovine serum that was not exposed to a hyaluronic acid gel sheet was used as a control group. The cytotoxicity results thus measured are listed in the following Table 1.

### Examples 5 to 8

The experiment was performed in the same manner as in Examples 1 to 4 except that 4.8 g of sodium hyaluronate was used such that hyaluronic acid had a concentration of 4.0 wt %.

### Examples 9 to 12

The experiment was performed in the same manner as in Examples 1 to 4 except that the immersion in an acetic acid anhydride solution was maintained at 5° C. for 72 hours.

### Examples 13 to 16

The experiment was performed in the same manner as in Examples 1 to 4 except that the primarily obtained hyaluronic acid gel sheet was immersed in a propionic anhydride solution for 24 hours.

### Comparative Example 1

The experiment was performed in the same manner as in Examples 1 to 4 except that the immersion in an acetic acid anhydride solution, the enhancement in a vacuum oven, and drying were omitted, and only a primary gel sheet was prepared and the cytotoxicity test was not performed.

### Comparative Example 2

The experiment was performed in the same manner as in Examples 1 to 4 except that the primarily obtained hyaluronic acid gel sheet was immersed in pure acetic acid, and the cytotoxicity test was not performed.

### Comparative Example 3

The experiment was performed in the same manner as in Examples 5 to 8 except that the primarily obtained hyaluronic acid gel sheet was immersed in pure acetic acid, and the cytotoxicity test was not performed.

### Comparative Example 4

The experiment was performed in the same manner as in Examples 13 to 16 except that the primarily obtained hyaluronic acid gel sheet was immersed in pure acetic acid, and the cytotoxicity test was not performed.

### Comparative Example 5

The experiment was performed in the same manner as in Examples 1 to 4 except that the primarily obtained hyaluronic acid gel sheet was immersed in an acetic acid anhydride solution with a concentration of 100 vol %, the enhancement and drying in a vacuum oven were omitted, blow-drying was performed, and the cytotoxicity test was not performed.

TABLE 1

| | | | | Shape of | | |
|---|---|---|---|---|---|---|
| | | | | gel sheet | | |
| | | | | after 168 | | |
| | | | | hours | | |
| | | | | ◯: | | |
| | | | | maintained | | |
| | | | | Δ: | | |
| | | | | partially | | |
| | | | | degraded | | |
| | Hyaluronic | | | X: fully | | Cytotoxicity |
| | acid | | Anhydride | degraded | | ◯: observed |
| | concentration | Anhydride and unit | concentration | pH | pH | X: not |
| | (wt %) | organic acid | (vol %) | 5.5 | 7.4 | Observed |
| Example 1 | 1.2 | Acetic acid | 100 | ◯ | ◯ | X |
| Example 2 | | anhydride/acetic | 50 | ◯ | ◯ | X |
| Example 3 | | acid | 20 | ◯ | ◯ | X |
| Example 4 | | | 5 | ◯ | ◯ | X |
| Example 5 | 4.0 | Acetic acid | 100 | ◯ | ◯ | X |
| Example 6 | | anhydride/acetic | 50 | ◯ | ◯ | X |
| Example 7 | | acid | 20 | ◯ | ◯ | X |
| Example 8 | | | 5 | ◯ | ◯ | X |
| Example 9 | 1.2 | Acetic acid | 100 | ◯ | ◯ | X |
| Example 10 | | anhydride/acetic | 50 | ◯ | ◯ | X |
| Example 11 | | acid | 20 | ◯ | ◯ | X |
| Example 12 | | | 5 | ◯ | ◯ | X |
| Example 13 | 1.2 | Propionic | 100 | ◯ | ◯ | X |
| Example 14 | | anhydride/propionic | 50 | ◯ | ◯ | X |
| Example 15 | | acid | 20 | ◯ | ◯ | X |
| Example 16 | | | 5 | ◯ | ◯ | X |
| Comparative | 1.2 | — | — | X | X | — |
| Example 1 | | | | | | |
| Comparative | 1.2 | Acetic acid 100% | 0 | X | X | — |
| Example 2 | | | | | | |
| Comparative | 4.0 | Acetic acid 100% | 0 | X | X | — |
| Example 3 | | | | | | |
| Comparative | 1.2 | Propionic acid | 0 | X | X | — |
| Example 4 | | 100% | | | | |
| Comparative | 1.2 | Acetic acid | 100 | ◯ | Δ | — |
| Example 5 | | anhydride/acetic | | | | |
| | | acid | | | | |

The gel sheet prepared according to an embodiment of the present invention can maintain its shape for a long time, exhibit improved mechanical strength, and have a low biodegradation rate due to not being dissolved in an aqueous solution with a pH from 5.5 to 8.5 that is a common in vivo condition, a physiological saline solution or a buffer solution, and high biocompatibility.

While the present invention has been particularly described with reference to exemplary embodiments, it will be understood by those skilled in the art that various changes in form and details may be made without departing from the spirit and scope of the present invention. Therefore, the exemplary embodiments should be considered in a descriptive sense only and not for purposes of limitation. The scope of the invention is defined not by the detailed description of the invention but by the appended claims, and encompasses all modifications and equivalents that fall within the scope of the appended claims.

What is claimed is:

1. A gel sheet, prepared by immersing a primary hyaluronic acid gel sheet, which is prepared by drying a flat plate on which an aqueous solution obtained by dissolving an alkali salt of high-molecular-weight hyaluronic acid in an alkaline solution is applied in a vacuum oven, in a crude solution of a low-molecular-weight organic acid anhydride or an anhydride solution diluted with a unit organic acid forming the anhydride, and secondarily drying the immersed primary hyaluronic acid gel sheet in the vacuum oven.

2. The gel sheet of claim 1, wherein the anhydride solution diluted with a unit organic acid forming the anhydride has a concentration (v/v) in a range of 5 to 100 vol %.

3. The gel sheet of claim 1, wherein the immersion step is performed at 4 to 70° C. for 2 hours or more.

4. The gel sheet of claim 1, wherein the anhydride includes acetic anhydride, propionic anhydride, butyric anhydride, caproic anhydride and isobutyric anhydride.

5. The gel sheet of claim 1, wherein the vacuum oven has a predetermined degree of vacuum and a predetermined temperature before the secondary drying.

6. The gel sheet of claim 1, wherein the immersed primary hyaluronic acid gel sheet is transferred into an incubator maintained at 40° C. before the secondary drying.

7. The gel sheet of claim 5, wherein the predetermined degree of vacuum is 100 Torr or less.

8. The gel sheet of claim 1, wherein the temperature of the vacuum oven and a primary drying time is gradually increased during the primary drying.
